(19) United States
(12) Patent Application Publication
(10) Pub. No.: US 2009/0105623 A1
(43) Pub. Date: Apr. 23, 2009

(54) ANKLE SPLINT WITH A PRONATION BELT HAVING A MASSAGING FLUID CUSHION

(75) Inventor: Erich ALBRECHT, Neubeuern (DE)

Correspondence Address: NIXON & VANDERHYE, PC, 901 NORTH GLEBE ROAD, 11TH FLOOR, ARLINGTON, VA, 22203, US

(73) Assignee: ALBRECHT GMBH, Neubeuern (DE)

(21) Appl. No.: 12/252,539

(22) Filed: Oct. 16, 2008

(30) Foreign Application Priority Data
Oct. 18, 2007 (DE) 10 2007 049 953.3

(51) Int. Cl. A61F 5/00

(52) U.S. Cl. 602/27

(57) ABSTRACT

An ankle splint has a pronation belt (11) with a sole section (17) and a lateral/instep section (18) an air cushion (12) designed as a continuous massaging cushion being provided in these sections of the pronation belt (11). The air cushion (12) is designed so that the fluid contained in the air cushion (12) is pumped dynamically out of the sole section (17) into the lateral/instep section (18) and vice versa.

The invention relates to an ankle splint with a pronation belt according to the preamble of Patent claim 1.

Ankle splints (ankle orthotic devices) in the form of functional U-shaped splints have proven successful in rehabilitation of injuries and/or degenerative diseases of the malleolar joint. Such ankle splints have relatively stiff side parts, which can be applied to the medial and lateral sides of the calf and attached there, e.g., by means of hook-and-loop closures, which are passed around the side parts in a circular pattern. The side parts here extend over the malleolar joint and downward into an area of the toot near the sole, where they are joined together by means of a cross strip which is passed beneath the heel. Such U-shaped splints support the malleolar joint medially and laterally, i.e., on both sides, while allowing extensive flexion and extension of the malleolar joint so that there can be a normal movement sequence.

With certain injuries in the ankle area, in particular injuries to the ligamentous apparatus of the ankle and when the ankle is already relaxed, it may be desirable to use, in addition to the ankle splint, a pronation belt which relieves the stress on the exterior ligaments of the ankle by lifting the forefoot and counteracting supination of the foot. Use of such a pronation belt is customary even in conservative or functional follow-up care for malleolar fractures and ruptured ligaments. The pronation belt then usually extends from the medial side part of the ankle splint beneath the sole of the foot outward and from there over the outer edge of the foot, over the back of the foot (instep) and back to the medial side part.

Starting from such a state of the art, the object of the present invention is to create an ankle splint having a pronation belt of the type defined above with which the regeneration of the foot, in particular in the area of the exterior ligaments, can be supported in an especially efficient manner.

This object is achieved according to the present invention by an ankle splint having the features of claim 1. Advantageous embodiments of the invention are described in the additional claims.

With the inventive ankle splint, the sole section and lateral/instep section of the pronation belt have a fluid cushion with a fluid filling, said fluid cushion being designed as a continuous massaging cushion arranged in both sections, such that at least a portion of the fluid contained in the fluid cushion is pumped into the lateral/instep section with compression of the sole section and is pumped into the sole section when the pressure on the sole section is released and the lateral/instep section is compressed, so that the fluid cushion experiences a dynamic change in thickness in the area of the sole of the lateral outside of the foot and n the area of the back of the foot.

With the help of the inventive fluid cushion, which may be designed as an air cushion in particular but also as a liquid cushion, the fluid contained in the fluid cushion is pumped alternately from the sole section upward into the lateral/instep section of the pronation belt and from there back down into the sole section. This is accomplished easily by the fact that the sole section is compressed when the person's foot is placed on the floor and the sole section is released again when the person's foot is lifted up from the floor. The fluid flowing back and forth produces an ongoing dynamic change in pressure in the areas of the foot in contact with the fluid cushion. This leads to an intense massaging effect extending from the sole of the foot over the outside edge laterally of the foot into the back of the foot, thereby intensely stimulating the circulation in this area and loosening the tissue. This also makes it possible in particular to prevent vascular congestion in these areas of the foot. The massaging fluid cushion thus has very positive effects on regeneration of damaged tissue. In addition, the massaging effect has a relaxing effect, so that comfort in wearing the pronation belt is increased.

According to an advantageous embodiment, the fluid cushion extends over the lateral half of the sole of the mid-foot and from there to the upper edge of the back of the foot. Alternatively, however, it is also conceivable here for the fluid cushion in the sole section to extend further inward medially, e.g., as far as the medial edge of the sole of the foot.

According to an advantageous embodiment, the fluid cushion consists of two plastic films arranged one above the other and joined together at the edges in a fluid-tight manner, having at least one connecting seam running in the longitudinal direction of the fluid cushion in the area of the lateral/instep section, so that the fluid cushion is subdivided in this area into at least two partial chambers that are separated from one another at the side but are continuous in the longitudinal direction of the pronation belt. In this way, the direction of the dynamic wave movement of the fluid can be influenced because the fluid no longer flows unhindered over the entire width of the fluid cushion in the transverse direction due to the joining seams but instead flows mainly in the longitudinal direction of the fluid cushion.

The invention is explained in greater detail below on the basis of the drawings as an example.

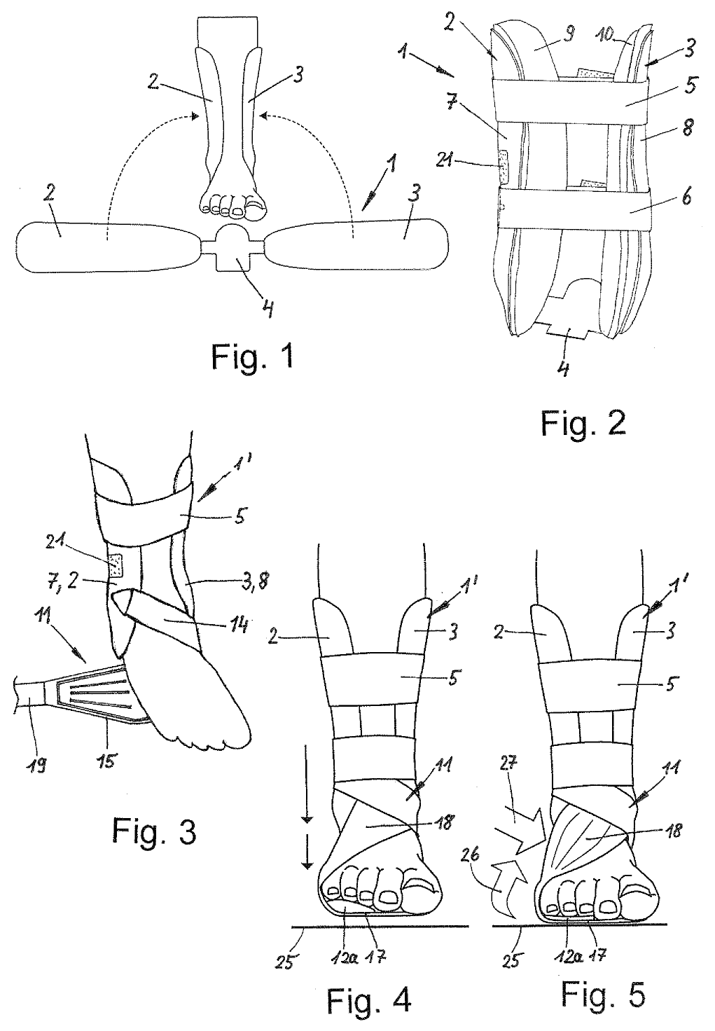

FIG. 1 shows a conventional ankle splint in a spread-apart state and after being applied to the calf,

FIG. 2 shows a diagram of a conventional ankle splint in its position alone in a state such as that in which it is usually applied to the calf,

FIG. 3 shows an ankle splint applied to a patient's foot with an inventive pronation belt, shown in shortened form, in an intermediate contact position,

FIG. 4 shows the inventive ankle splint in the applied state, whereby the foot is lifted up from the floor,

FIG. 5 shows a diagram according to FIG. 4, where the foot is placed on the floor,

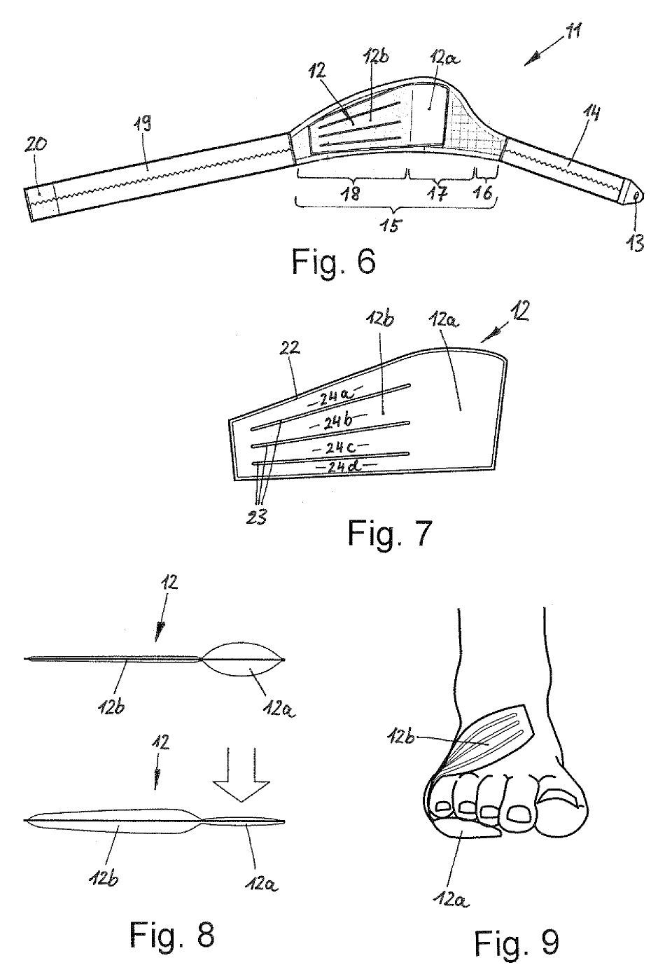

FIG. 6 shows a top view of the inventive pronation belt in its position standing alone,

FIG. 7 shows a top view of the fluid cushion of the pronation belt from FIG. 6, standing alone,

FIG. 8 shows two side views of the fluid cushion to illustrate the pumping effect, and

FIG. 9 shows a diagram of the fluid cushion applied to the foot, standing alone.

FIGS. 1 and 2 show a conventional ankle splint 1 comprising a lateral side part 2 and a medial side part 3, joined together at their lower ends by means of a sole strip 4 that can be passed beneath a patient's heel. The side parts 2, 3 may be applied laterally and medially to the foot and/or to the lower portion of a patient's calf, as shown in FIG. 1, where the sole strip 4 is arranged beneath the sole of the foot, and the lateral side part 2 extends from just above the sole over the lateral area of the upper ankle joint to a side area of the calf, while the medial side part 3 extends from just above the sole over the medial side of the upper ankle joint to a medial side area of the calf.

Side parts 2 and 3 are separate parts which are joined together only in the lower end area by means of the sole strip 3 that is flexible but has good tensile strength. Because of this design, which is fundamentally already known, such splints are also known as U-shaped splints. The side parts 2, 3 may therefore be pivoted apart from one another to apply the ankle splint to the leg, so that the foot can be inserted between them, and the side parts 2, 3 can easily be placed laterally and/or medially in contact, as shown in FIG. 1.

In the applied state, the side parts 2, 3 are wrapped in a circular manner about both sides in the familiar way by using an upper shell belt 5. The upper shell belt 5 is expediently situated in the upper end area of the two side parts 2, 3 and is connected at one end securely to one of the two side parts, preferably to the lateral side part, while the other end can be attached by means of a hook-and-loop closure.

As shown in FIG. 2, with conventional ankle splints 1 beneath the upper shell belt 5, a lower shell belt 6 may be wrapped in a circular manner around the two side parts 2, 3 in the same way as the upper shell belt to tighten the two side parts 2, 3 against one another just above the ankle and thereby hold them against the patient's leg with the desired strength.

The side parts 2, 3 usually have shell parts 7, 8 that are made of plastic and are designed to be relatively rigid, so that they can support the ankle appropriately. The curvature of the shell parts 7, 8 is adapted to the shape of the leg to achieve maximum wearing comfort and to prevent pressure points. In addition, the shell parts 7, 8 are lined with padding 9, 10 in the usual way on the side adjacent to the leg, this padding optionally comprising an air chamber padding.

In contrast with the known ankle splint 1 shown in FIG. 2, the inventive ankle splint 1′ shown in FIGS. 3 to 5 does not have a lower shell belt 6, which is merely wrapped around the two side parts 2, 3 in a circular manner, but instead has a special pronation belt 11, which is shown in the individual diagram in FIG. 6. Pronation belts are known for lifting a patient's forefoot to relieve the exterior ligaments in particular. However, in contrast with known pronation belts, the inventive pronation belt 11 has a special fluid cushion 12, which exerts a dynamic pumping effect on the adjacent foot areas in walking and thus acts as a massage cushion.

As shown in FIGS. 3 to 6, pronation belt 11 has a belt lock with a fastening element 13 on one end with which the pronation belt 11 can be detachably attached in the lower half of the lateral side part 2. This fastening element 13 may be, for example, a T-shaped anchor element protruding laterally beyond the pronation belt 11, which may be inserted through an elongated opening in the lateral shell part 7, so that after a pivoting movement of the pronation belt 11, it engages under the shell part 7 from the inside. Other possible fastening means are also conceivable, however, e.g., hook-and-loop closures. It is also possible to fixedly attach the pronation belt 11 to the lateral shell part 7.

A belt section 14 having good tensile strength is connected to the belt lock. This belt section 14 is passed diagonally over the back of the foot and down to the medial side area of the mid-foot, as shown in FIGS. 3 to 5.

As shown in FIG. 6, a belt section 15 which consists of a medial section 16, a sole section 17 and a lateral/instep section 18 is attached to the belt section 14 having good tensile strength. In the applied state of the pronation belt 11, the medial section 16 is in contact with the medial side of the foot, while the sole section 17 is situated beneath the sole of the foot. The lateral/instep section 18 extends from the lateral outside of the mid-foot upward to the upper end of the back of the foot. In particular in the area of the sole section 17 and lateral/instep section 18, the pronation belt 11 is widened, having its greatest width in the area of the sole section 17 adjacent to the lateral/instep section 18 in the exemplary embodiment shown here. At least partial areas of the belt section 15 are preferably designed to be elastic.

A belt section 19 having good tensile strength is connected to the belt section 15 and it has a uniform width in the same way as does belt section 14. In applying the pronation belt 11, the belt section 19 is first passed from the position shown in FIG. 3 beneath the belt section 14 and then passed medially about the medial side part 3 dorsally and from there around the lateral side part 2 back to the area of the medial side part 3, where it is secured at its end by means of a hook-and-loop closure 20 (FIG. 6) to the layer of the belt section 19, which is already present. FIG. 3 also shows a hook-and-loop element 21, which is attached to the lateral side part 2 over which the belt section 19 is passed so that it is also additionally secured there by hook and loop.

As shown in FIG. 6 in particular, the fluid cushion 12 is arranged in the area of the sole section 17 and the lateral/instep section 18 of the pronation belt 11. The fluid cushion 12 is expediently embedded between elastic textile layers of the belt section 15, so that it is secured in a slip-free manner in the pronation belt 11 and is additionally protected from damage. The fluid cushion 12 is adapted to the outer contour of the sole section 17 and the lateral/instep section 18, as shown in FIG. 6, and extends over the lateral half of the sole section 17 and up to the vicinity of the opposing end of the lateral/instep section 13. The fluid cushion 12 thus consists of a sole cushion section 12a and a lateral/instep cushion section 12b that is connected directly to the sole cushion section 12a and is designed in one piece with it. The width of the fluid cushion 12 is only slightly less than that of the sections 17, 18.

The fluid cushion 12 consists of two flexible plastic films that are arranged one above the other and are welded or glued together in a fluid-tight manner at the edges, enclosing a fluid chamber. Within the fluid chamber there is a predetermined amount of fluid, such that the amount of fluid is such that the plastic films are pressed slightly apart. As also shown in FIG. 7 in particular, the lateral/instep cushion section 12b has joining seams 23 with which the plastic films are welded together. The joining seams 23 run in the longitudinal direction of the fluid cushion 12 up to the vicinity but not entirely up to the end of the fluid cushion 12, which is adjacent to the belt section 19. In the present exemplary embodiment, the fluid chamber is subdivided into four elongated subchambers 24a, 24b, 24c, 24d by the three joining seams 23, which are arranged side-by-side but are separated from one another at the sides. In their two end areas, these subchambers are in fluid connection with one another. In the area of the sole cushion section 12a, the fluid cushion 12 does not have any joining seams 23, however.

Owing to the design and arrangement of the fluid cushion 12 described here, when the foot is placed on the floor 25, the sole cushion section 12a of the fluid cushion 12, which is beneath the sole of the foot, is compressed so that the fluid is forced out of this section and pumped into the adjacent lateral/instep section 12b. This procedure is illustrated by the arrow 26 in FIG. 5 and by the lower diagram in FIG. 8. In this way, the lateral/instep section 12b is widened and/or thickened, so that an increased compressive force is applied to the adjacent part of the foot, as illustrated by the arrow 27 in FIG. 5. When the foot is lifted up from the floor 25 again, the fluid flows back down into the sole cushion section 12a due to the pressure differences prevailing within the fluid cushion 12, so the pressure on the lateral side and the back of the foot is released again. Thus walking causes a dynamic and constantly changing compressive force to be applied to the lateral side areas of the foot up to the upper edge of the back of the foot, so that fluid cushion 12 can act as a massage cushion in this entire foot area.

1. An ankle splint with side parts supporting the ankle of a patient, said side parts being applicable to the medial and lateral sides of the calf and attachable thereto, and with a pronation belt that can be passed around the foot for relieving the stress on the exterior ligaments of the ankle by lifting the forefoot, such that the pronation belt has a sole section that can be guided across and beneath the sole of the foot and, connected thereto, has a lateral/instep section that can be passed over the lateral outside of the foot and the back of the foot, characterized in that the sole section and the lateral/instep section of the pronation belt have a fluid cushions, which is designed as a continuous massage cushion arranged in both sections and having a fluid filling, such that at least a portion of the fluid contained in the fluid cushion is pumped into the lateral/instep section when the sole section is compressed and is pumped into the sole section when the pressure on the sole section is released and the lateral/instep section is compressed, so that the fluid cushion experiences a dynamic change in thickness in the area of the sole of the foot, the lateral outside of the foot and the back of the foot.

2. The ankle splint according to claim 1, characterized in that the fluid cushion extends over the lateral half of the sole of the mid-foot and from there to the upper end of the back of the foot.

3. The ankle splint according to claim 1, characterized in that the fluid cushion consists of two plastic films arranged one above the other and joined together in a fluid-tight manner, having at least one joining seam running in the longitudinal direction of the fluid cushion in the area of the lateral/instep section, such that the fluid cushion has at least two subchambers in this area that are separated from one another laterally but are continuous in the longitudinal direction of the pronation belts.
